(19) United States
(12) Patent Application Publication
(10) Pub. No.: US 2015/0216898 A1
(43) Pub. Date: Aug. 6, 2015

(54) COMPOSITION FOR TREATING PSORIASIS

(71) Applicant: PSORIASIS+CREAMS SWEDEN AB, Halmstad (SE)

(72) Inventor: Ana Moll Lorca, Palma de Mallorea (ES)

(73) Assignee: PSORIASIS+CREAMS SWENDEN AB, HALMSTAD (SE)

(21) Appl. No.: 14/426,277

(22) PCT Filed: Sep. 3, 2013
(86) PCT No.: PCT/EP2013/068175

§ 371 Date: Mar. 5, 2015

(30) Foreign Application Priority Data
Sep. 5, 2012 (EP) 12183189.5

(51) Int. Cl. A61K 35/04; A61K 9/00; A61K 31/27; A61K 31/17; A61K 31/573; A61K 31/4402

(52) U.S. Cl. CPC A61K 35/04; A61K 31/573; A61K 31/4402; A61K 31/27; A61K 31/17; A61K 9/0014

(57) ABSTRACT

A dermal composition for treating or soothing psoriasis lesions, wherein the composition comprises a tar extract, a corticosteroid, an antipruritic, an antibiotic, urea, an absorption base, water, lanolin, and propylene glycol.

### TECHNICAL FIELD

The present invention relates to a novel composition for treating psoriasis and a kit comprising such a composition and a second composition having emollient, lubricating and moisturizing effect on the skin. The first composition comprises various active ingredients including a corticosteroid.

### BACKGROUND

Psoriasis is an autoimmune disease that affects the skin. The name psoriasis has its basis in Ancient Greek, psoriasis roughly meaning “itching condition”. There are five types of psoriase i.e. plaque, guttate, inverse, pustular, and erythrodermic. Plaque psoriasis (psoriasis vulgaris) is the most common one. Plaque psoriasis is commonly seen as red and white hues of scaly patches appearing on the top first layer of the epidermis (skin). Lesions frequently occur on the skin of the elbows and knees. It can however affect any area, including the scalp, palms of hands and soles of feet, and genitals.

The disorder is seen as a chronic recurring condition whose severity varies from minor localized lesions to complete body coverage. Its cause is not fully understood, but it is believed to have a genetic component and local psoriatic changes can be triggered by an injury to the skin known as the Koebner phenomenon. Further, various environmental factors have been suggested as aggravating to psoriasis. Those include stress, withdrawal of systemic corticosteroid, as well as other environmental factors.

While there are many treatments available, psoriasis is a challenge to treat because of its chronic recurrent nature. Further, as the cause of psoriasis is not fully understood, the treatment typically involves alleviating the manifesting symptoms rather than treating the underlying disease. As immunosuppressant medications, such as corticosteroids, can clear, or at least soothe, psoriasis lesions, one of the hypotheses about the process that occurs in the development of the disease is that the disease is an immune-mediated disorder. The excessive reproduction of skin cells is believed to be secondary to factors produced by the immune system.

Despite that the corticosteroid used to treat lesions typically are low-strength corticosteroids, several side effects are usually seen. The corticosteroid causes skin damage, such as skin thinning, changes in pigmentation, easy bruising, stretch marks, redness and dilated surface blood vessels; especially after prolonged use. As psoriasis causes itching, the subject often scratches and thereby damages the sensitive skin. The cuts may further become infected by bacteria, fungi etc.; especially as the corticosteroid is anti-inflammatory, thereby affecting the immune system and delaying its response to microorganisms. Thus, while corticosteroids are effective in treating psoriasis lesions, the treatment still suffers from various drawbacks. Commonly, the psoriasis lesions reoccur shortly after withdrawal of topical corticosteroids.

The treatment of psoriasis also includes the use of bath solutions and moisturizers, mineral oil, and petroleum jelly as they may help to soothe affected skin and reduce the dryness, which accompanies the build-up of skin on psoriatic plaques.

Further medicated creams and ointments, which are applied directly to psoriatic plaques, may be used to treat psoriasis. Such creams and ointments may reduce the underlying inflammation, remove built-up scale, reduce skin turn over, and clear affected skin of plaques. They contain an active component, such as coal tar, dithranol (anthralin), corticosteroids, vitamin D3 analogues (for example, calcipotriol), and retinoids.

As an example, US 2007/207222 discloses a composition including a wax and a therapeutically effective amount of tar for topical treatment of a tar-responsive dermatological disorder, the composition being in liquid or light gel form when at a temperature selected from room temperature and a temperature of skin of a mammal upon application of the composition to the skin of the mammal.

Further, use of tar comprising composition has, for example, been disclosed by Paghdal K V et al “Topical tar: Back to the future”, in Journal of the American Academy of Dermatology, vol. 61(2) 2009, p. 294-302, and by Goodfield M et al: “Double-blind, randomised, multicentre, parallel group study comparing a 1% coal tar preparation (Exorex) with a 5% coal tar preparation (Aiphosyl) in chronic plaque psoriasis” Journal of dermatological treatment, vol. 15(1) 2004 p. 14-22.

However, there is a need in the art for an effective composition comprising a corticosteroid for treating psoriasis.

### SUMMARY

The present invention seeks to mitigate, alleviate, circumvent or eliminate at least one, such as one or more, of the above-identified deficiencies by providing a dermal composition comprising a tar extract, such as saponified coal tar; a corticosteroid, such as clobetasol propionate (i.e. [17-(2′-chloroacetyl)-9-fluoro-11-hydroxy-10,13,16-trimethyl-3-oxo-6,7,8,11,12,14,15,16- ctahydrocyclopenta[a]phenanthren-17-yl] propanoate); an antipruritic, such as an antihistamine, e.g. Chlorpheniramine (i.e. 3-(4-chlorophenyl)-N,N-dimethyl-3-pyridin-2-yl-propan-1-amine); an antibiotic, such as an anti-fungal agent, e.g. tolnaftate (i.e. O-2-naphthyl methyl(3-methylphenyl)thiocarbamate); urea; an absorption base; water; lanolin; and propylene glycol. Further, the composition may comprise glycerin, a vegetable oil, such as sweet almond oil, ammonium lactate, and/or a preservative.

Another aspect of the invention relates to use of such a composition for treating or soothing psoriasis lesions by applying the composition to areas of the skin affected by psoriasis.

Another aspect of the invention relates to a kit comprising two distinct compositions, the first composition being such a composition as described herein above, and the second composition being composition comprising aqueous hyaluronic acid, an absorption base, and vegetable oil. Further, the second composition may comprise vitamin E, and optionally a fragrance. The vegetable oil may comprise comprises almond oil, argan oil, and/or macadamia oil.

Another aspect of the invention relates to use of such a kit for treating or soothing psoriasis lesions.

Further advantageous features of the invention are defined in the dependent claims. In addition, advantageous features of the invention are elaborated in embodiments disclosed herein.

### DETAILED SUMMARY OF PREFERRED EMBODIMENTS

The present inventor has devised a dermal composition for treating or soothing psoriasis lesions comprising a corticosteroid and further active ingredients including a tar extract, an antipruritic, an antibiotic and urea. It was found that such a composition was very effective in treating or soothing psoriasis lesions, as the ones seen in subjects suffering from psoriasis vulgaris. Further, it was found that the subjects did experience less severe side effects than experienced previously with conventional compositions comprising a corticosteroid as the active ingredient. Without being bound to any theory is believed that active ingredients act in a synergistic manner. Especially, it seems that side effects typically seen with corticosteroid (e.g. skin damage, such as skin thinning, changes in pigmentation, easy bruising, stretch marks, redness and dilated surface blood vessels) are alleviated even if a potent corticosteroid is used. The time period for symptom relief was significantly shorter than the one experienced with a conventional composition comprising a corticosteroid. Further, the subjects remained asymptomatic for a prolonged period of time. The same advantages were confirmed also in comparing the present composition with combined treatment with corticosteroids and tar extract supplemented with an emollient. While the mechanism underlying the synergistic mode of action still is to be revealed, a synergistic mode of action clearly is at hand.

An embodiment thus relates to a dermal composition comprising a tar extract, a corticosteroid, an antipruritic, an antibiotic and urea. Further, the dermal composition comprises water, lanolin, and propylene glycol. In order to obtain a dermal composition to be applied to the skin, the composition also comprises an absorption base such that a stable emulsion may be obtained.

The corticosteroid is preferably a potent corticosteroid, such as clobetasol propionate, betamethasone dipropionate, halobetasol propionate, fluocinonide, diflorasone diacetate, mometasone furoate, halcinonide, or desoximetasone. According to an embodiment the corticosteroid is clobetasol propionate. While less potent corticosteroids typically are used in treating psoriasis topically, it was found that the skin tolerated potent corticosteroids well, when administered in the present composition. Further, it was found that potent corticosteroids were effective in treating or soothing psoriasis lesions.

Further, the dermal composition comprises a tar extract. Tar, or tar extract, is obtained as a by-product by dry distillation of organic materials, such as coal or wood in the absence of oxygen. There are different types of tar extracts including coal tar, wood tar and shale tar. In the present composition, the tar extract is typically a coal tar extract, also known as liquor carbonis detergens. Especially, the coal tar extract may be saponified coal tar. The tar extract provides the composition with keratolytic and antiseptic properties, whereby counteracting the skin damaging effect of the corticosteroid. Further, the antiseptic effect of the tar extract may prevent the sensitive skin from becoming infected.

The properties of the present composition could however not merely be explained by the incorporation of a tar extract, as the composition provides superior results compared to combined treatment with corticosteroids and tar extract supplemented with a emollient. While the exact mechanism remains to be revealed, clearly also other constituents of the present composition are essential for the synergistic effect provided.

The dermal composition also comprises urea. Urea does not influence the emulsion's stability, but it is important for its therapeutic action. Urea provides the composition with moisturizing and keratolytic properties. Further, it reduces itching and hyperkeratosis, which both common symptoms of psoriatic plaques. By including urea in the composition, it is believed that the side effects of the corticosteroid may be alleviated. While urea is a preferred constituent, it may be replaced with salicylic acid, also known to have keratolytic properties. However, urea is preferred over salicylic acid, as salicylic acid may provoke skin hypersensitivity.

While urea may act as an antipruritic, it preferred to include an additional antipruritic in the composition. One common symptom of psoriasis is itching. As the skin may be sensitive to scratching, especially if being treated with a corticosteroid, cuts and subsequent infections may be counteracted by including an antipruritic in the composition. The antipruritic may be antihistamine, such as chlorpheniramine. Typically the antihistamine is a H₁-receptor antagonist. Except for chlorpheniramine there are various other examples of H₁-receptor antagonists, which may be included in the composition, such as diphenhydramine, thonzylamine, mepyramine, tenalidine, tripelenamine, chloropyramine, promethazine, tolpropamine, isothipendyl, and chlorphenoxamine.

Although, the skin damaging effect of the corticosteroid is being counteracted, the skin is anyhow sensitive and susceptible to infections. In order to counteract infections and to assist the skin in recovering, the composition comprises an antibiotic, such as fungicide or bactericide. While the tar extract provides the composition with antiseptic properties, it was anyhow found to be advantageous to include an antibiotic in the composition.

According to an embodiment the antibiotic is an anti-fungal agent, e.g. a fungicide. The fungicide is typically a fungicide being pharmaceutical acceptable for topical administration to the skin. A preferred example is tolnaftate. Except for tolnaftate there are various examples of fungicides which may be included in the composition, such as amorolfin, butenafine, naftifine, and terbinafine. Further the fungicide may be an imidazole, such as Bifonazole, Butoconazole, Clotrimazole, Econazole, Fenticonazole, Isoconazole, Ketoconazole, Miconazole, Omoconazole, Oxiconazole, Sertaconazole, Sulconazole, and Tioconazole, a triazole, such as albaconazole, fluconazole, Isavuconazole, Itraconazole, Posaconazole, Ravuconazole, Terconazole, and Voriconazole, or a thiazole such as Abafungin. The fungicide may also be a polyene macrolide fungicide, such as natamycin and nystatin.

Common medicaments of psoriasis include various ointments, creams, solutions and moisturizers. Typically a subject suffering from psoriasis typically has to apply various compositions in order to alleviate the disease. As several compositions are to be applied, the compliance may be low. As the disorder manifests it self by various symptoms over time and as the primary skin condition may give rise to secondary skin infections caused by bacteria and fungi, the disorder is complex to treat and manage. However, it is very difficult to formulate various components, having different physical properities (solubility, melting point, viscosity, etc.), into a single stable composition.

In order to provide a single formulation, the present composition is preferably formulated as an emulsion, as hydrophilic as well as hydrophobic constituents are to be present. Thus, the composition comprises an absorption base and water forming the basis for the emulsion. Further, it comprises propylene glycol. By including propylene glycol, constituents not readily soluble in water or oil may be included in the composition. Especially, propylene glycol assists in dispersing the main active ingredient, i.e. the corticosteroid, in the composition.

Further, the composition comprises lanolin. Lanolin acts emulsifying, emollient and moisturizing. More importantly, lanolin also stabilizes the emulsion, whereby coal tar may be added to the emulsion without disrupting its structure.

Absorption bases, such as oil/water absorption bases, are known in the art for making creams and ointments. An absorption base is an oleaginous base that contains an emulsifying agent. When water is taken up into the base, it will form an emulsion. Oil/water absorption bases typically can incorporate at least 45% of their volume of water. Water soluble ingredients may be added to the water phase of the emulsion, while non-water soluble ingredients may be added to the oil phase of the emulsion, provided that they are soluble in the oil phase. Insoluble constituents may be included mechanically into the emulsion.

According to a preferred embodiment, the absorption base is a self emulsifying oil/water absorption base. According to another embodiment, the absorption base is a water/oil absorption base. The emulsifying agent may be ionic as well as non-ionic. According to an embodiment, the emulsifying agent is non-ionic.

An example of a preferred absorption base is Neo-PCL O/W self emulsifying base. This absorption base comprises beeswax (Cera Alba), cetearyl, ethylhexanoate, stearyl alcohol, stearyl septanoate, cetyl palmitate, steareth-7, steareth-10, stearyl caprylate, myristyl alcohol.

Further the composition comprises water. By dispersing water in the absorption base an emulsion may be obtained. In order to prolong the shelf life of the composition it may further comprise a preservative, such as a paraben. Examples of suitable parabens are propyl 4-hydroxybenzoate and methyl 4-hydroxybenzoate.

According to an embodiment, the composition further comprises a vegetable oil, such as sweet almond oil. Sweet almond oil is a vegetable oil obtained by expression of the mature seeds of Prunus dulcis. It consists mainly of glycerides of oleic acid and smaller amounts of linoleic and palmitic acids. It has demulcent and emollient nutritional properties. Further and more importantly, a vegetable oil, such as sweet almond oil, was found to act synergistically with lanolin. By its own lanolin is not absorbed by the skin, but when mixed with a vegetable oil, such as sweet almond oil, it provides an emollient cream that penetrates the skin and facilitates the absorption of the active ingredients. Thereby, the composition softens the skin and assist in removing scale from psoriatic lesions more easily.

According to an embodiment, the composition further comprises ammonium lactate. Ammonium lactate is effective in preventing and treating dry skin. Further, it also helps to regenerate damaged skin. Without being bond to any theory, it is believed to contribute to restoring the skin and to counteract the skin damaging effects of the corticosteroid.

According to an embodiment, the composition further comprises glycerine. It may be used an adjuvant for the emulsion as it has lubricating, humectant and hygroscopic properties, which help the composition to stick to the skin and prevent water loss and subsequent drying of the emulsion over time. Further, it provides the composition with an emollient effect which in turn may improve the psoriatic lesion by softening the skin.

The various constituents may be present in the composition in various amounts. According to an embodiment, the composition comprises:

5 to 10 wt % of a tar extract;

0.01 to 2 wt % of a corticosteroid;

0.01 to 2 wt % of an antipruritic;

0.5 to 5 wt % of an antibiotic;

5 to 15 wt % urea;

10 to 30 wt % of an absorption base;

30 to 50 wt % water;

1 to 5 wt % lanolin;

1 to 10 wt % propylene glycol;

optionally, 1 to 10 wt % vegetable oil;

optionally 5 to 15 wt % of an aqueous solution of ammonium lactate, such as aqueous solution of ammonium lactate comprising about 70 wt % ammonium lactate; and

optionally 1 to 10 wt % glycerin.

As already described the composition is effective in treating or soothing psoriasis lesions; especially lesions seen at subjects suffering from psoriasis vulgaris. According to an embodiment it is used for said purpose. Further, the composition may be used in a method of treating or soothing psoriasis lesions. According to another embodiment, the composition is used in the manufacture of a medicament for treating or soothing psoriasis lesion. When being used to treat or soothe psoriasis lesions, the composition is applied to areas of the skin affected by psoriasis. As the corticosteroid may damage the skin, it is preferred to avoid applying the composition to areas of the skin not affected by psoriasis. Typically, the composition is applied once a day in the evening prior to sleep. As the composition comprises a corticosteroid, the treatment is to be discontinued, once complete symptom relief has been accomplished. Symptom relief is typically seen within a week, implying the itching ceases, the scaling starts to decrease, etc. Complete symptom relief is typically seen within 1 to 3 months.

Subjects with severe psoriasis, previously not having been asymptomatic despite various types of treatments, including topical corticosteroids, typically unexpectedly experience complete symptom relief within 3 months. Further, they do usually remain asymptomatic for at least 6 months after withdrawal of the composition.

While the first composition comprises constituents counteracting the skin damaging effect of the corticosteroid, it may anyhow be advantageous to apply a second composition for minimizing the skin damaging effect of the corticosteroid. Typically, the first composition is very effective in removing the psoriasis lesions, whereby prolonged treatment not is necessary and very few side effects are thus seen during the treatment. However, in cases of severe psoriasis it may become necessary to prolong the treatment. Especially in such cases, it may be advantageous to apply a second composition for minimizing the skin damaging effects of the corticosteroid. According to an embodiment, such a second composition comprises an aqueous solution of hyaluronic acid, an absorption base and vegetable oil. The absorption base may of the same kind as the one used in the first composition.

An embodiment thus relates to kit comprising such a first composition for treating or soothing psoriasis lesions as already have been described herein, and a second composition comprising aqueous hyaluronic acid, an absorption base and vegetable oil. Typically, the compositions are applied separately; the first composition being administered once a day in the evening prior to sleep and the second one once a day in the morning.

The second composition has an emollient, lubricating and moisturizing effect on the skin. Furthermore and importantly, the second composition, due to the presence of hyaluronic acid, assists in regenerating and reconstructing collagen in the skin. Accordingly, also prolonged use of the first composition is possible without pronounced damages to the skin. It is to be observed that use of the second composition also may be advantageous in combination with first composition despite the treatment not being prolonged.

Furthermore, the second composition may also comprise vitamin E. The vegetable oil may comprise almond oil, argan oil, and/or macadamia oil. The second composition may also comprise a fragrance, such as peach essence.

As already described the first composition is effective in treating or soothing psoriasis lesions and the second one in minimizing the skin damaging effect of the corticosteroid. According to an embodiment the kit is used for treating or soothing psoriasis lesions. Further, the kit may be used in a method of treating or soothing psoriasis lesions. According to another embodiment, the kit is used in the manufacture of a medicament for treating or soothing psoriasis lesion. When being used to treat or soothe psoriasis lesions, the first composition is applied to areas of the skin affected by psoriasis. As the corticosteroid may damage the skin, it is preferred to avoid applying the first composition to areas of the skin not affected by psoriasis. Typically, the first composition is applied once a day in the evening prior to sleep, while the second composition is administered one once a day in the morning.

The second composition typically comprises 0.1 to 5 wt %, such as 0.5 to 2 wt %, hyaluronic acid. Further, it typically comprises 10 to 20 wt % of a vegetable oil or a mixture of vegetable oils.

Without further elaboration, it is believed that one skilled in the art can, using the preceding description, utilize the present invention to its fullest extent. The preferred specific embodiments described herein are, therefore, to be construed as merely illustrative and not limitative of the remainder of the description in any way whatsoever. Further, although the present invention has been described above with reference to specific embodiments, it is not intended to be limited to the specific form set forth herein. Rather, the invention is limited only by the accompanying claims and, other embodiments than the specific above are equally possible within the scope of these appended claims, e.g. different than those described above.

In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous.

In addition, singular references do not exclude a plurality. The terms “a”, “an”, “first”, “second” etc do not preclude a plurality.

### EXPERIMENTAL

The following examples are mere examples and should by no mean be interpreted to limit the scope of the invention. Rather, the invention is limited only by the accompanying claims.

### Example 1

### Night Cream

A composition comprising urea (100 g), saponified coal tar (70 ml), tolnaftate (20 g), aqueous (70%) ammonium lactate (100 ml), clobetasol dipropionate (5 g), chlorpheniramine maleate (6 g), propylene glycol (50 ml), lanolin (30 g), sweet almond oil (50 ml), neo-pcl o/w (175 g), glycerine (50 ), and aqua conservans (distilled water containing parabens: 0.1% sodium nipagin and 0.05% sodium nipasol) to 1000 g was prepared as indicated below.

In preparing the compositions the solid components were weighed and the liquids measured.

Firstly an aqueous phase and an oil phase were prepared.

Aqueous phase: The aqua conservans was measure in a test tube and poured into a 1-litre Erlenmeyer flask. Subsequently urea was added and the mixture was stirred until the urea had dissolved, whereupon chlorpheniramine maleate was added and the mixture was stirred until the chlorpheniramine maleate had dissolved. At last glycerine and an ammonium lactate solution was added to the aqueous solution.

Oil phase: Lanolin, Neo-PCL O/W and sweet almond oil was put into a wide-mouth 2-litre flask and mixed.

The flasks with the aqueous phase and the oil phase were both placed in a laboratory water bath at 60 to 70° C. until both phases had reach the same temperature (about 65° C.). At this temperature, both are in a liquid state.

Subsequently, the aqueous phase was gradually added to the oil phase under continuously stirring. Once both phases had been mixed, the resulting mixture was continuously stirred at constant speed until cooled to ambient temperature. The resulting mixture is an aqueous external phase emulsion (O/W emulsion).

Solid phase: Tolnaftate and clobetasol dipropionate were weighed into a mortar and mixed with a pestle to reduce their size into a fine dust. Then, propylene glycol was added and the resulting mixture mixed and grinded with pester until a homogeneous fluid mass was obtained.

Once a homogeneous fluid mass had been obtained, approximately 50 g of the aqueous external phase emulsion was added to the mortar and mixed with the homogeneous fluid mass. The resulting mixture was then added to the reaming part of the aqueous external phase emulsion. Finally, 100 ml saponified coal tar was measured into a test tube and mixed with the emulsion comprising the rest of the components using a mechanical stirrer to obtain the active night cream.

### Example 2

### Day Cream

A composition comprising 500 g of an aqueous solution of hyaluronic acid (2 vol %), comprising 10 vol % propylene glycol, vitamin E (α-tocopherol; 10 g), sweet almond oil (50 g), argan oil (50 g), macadamia oil (50 g), peach essence (a drop) and neo-pcl o/w to 1000 g was prepared as indicated below.

The aqueous solution of hyaluronic acid, comprising propylene glycol, was added to the Neo-PCL O/W and the resulting emulsion was stirred. Thereafter the oils were added. Then and α-tocopherol was added and at last peach essence. The day cream was obtained as a creamy emulsion.

### Example 3

### Treatment of Subjects Suffering From Psoriasis

The night cream was applied areas of the skin affected by psoriasis once a day prior to sleep. Care was taken to only apply the potent cream to affected areas. In the morning the following day, the day cream was applied to the skin. As the day cream has an emollient, lubricating and moisturizing effect on the skin it may be used also at parts of the skin being unaffected by psoriasis.

Subject 1

A male subject, age 57, having psoriasis lesions (approx. size 10-15 cm) covering approximately 10% of his body, and suffering from severe itching, did apply the night cream and day cream above for 3 months.

The subject had been diagnosed with psoriasis for nearly 20 years ago and had previously used combinations of local corticosteroids and emollient and moisturizing creams. Further, the subject had also tried photo therapy. However, at the best those treatments alleviated the lesion somewhat. Further, the subject experienced side-effects (e.g. skin damage, such as skin thinning, changes in pigmentation, easy bruising, stretch marks, redness and dilated surface blood vessels) typically seen with treatment with corticosteroids.

Already after 1 week of administration of the night and day cream, the lesions were significantly reduced. Further, the itching has ceased. After 3 months of treatment the symptoms of psoriasis was gone. Despite withdrawal of the treatment, no symptoms of psoriasis were seen for nearly a year. The subject had previously not experienced complete symptom relief with conventional treatments.

Subject 2

A male subject, age 59, having psoriasis lesions (approx. size 10-15 cm) covering approximately 2% of his body, and suffering from severe itching did apply the night cream and day cream above for 3 months.

The subject had been diagnosed with psoriasis for nearly 25 years ago and had previously used combinations of local corticosteroids and emollient and moisturizing creams. Further, the subject had also tried photo therapy as well as a topical tar based product (Skillingarydsalvan Original). However, at the best those treatments alleviated the lesion somewhat. Further, the subject experienced side-effects (e g skin damage, such as skin thinning, changes in pigmentation, easy bruising, stretch marks, redness and dilated surface blood vessels) typically seen with treatment with corticosteroids.

Already after 1 week of administration of the night and day cream, the lesions were significantly reduced. Further, the itching had ceased. After 3 months of treatment the symptoms of psoriasis was gone. Despite withdrawal of the treatment, no symptoms of psoriasis were seen for nearly a year

Subject 3

A male subject, age 62, having psoriasis lesions (approx. size 10-15 cm) covering approximately 3% of his body, and suffering from severe itching did apply the night cream and day cream above for 3 months.

The subject had been diagnosed with psoriasis for nearly 15 years ago and had previously used various types of treatments (e.g. emollient and moisturizing creams, photo therapy, topical tar based products etc.). However, at the best those treatments alleviated the lesions somewhat.

Already after 1 week of administration of the night and day cream, the lesions were significantly reduced. Further, the itching had ceased. After 3 months of treatment the symptoms of psoriasis was gone. Despite withdrawal of the treatment, no symptoms of psoriasis were seen for nearly a year.

Evaluation

As can be seen from the examples provided above, the night cream, especially if combined with the day cream, was efficient in removing psoriasis lesions. It is to be noted that the subjects previously had used compositions comprising corticosteroids as well as compositions comprising tar extracts, in treating their psoriasis, but with limited effect. Further, the side effects typically seen with potent corticosteroid was significantly alleviated and the subjects did actually experience less severe side effect than the ones previously experienced with low-strength corticosteroid.

Example 4

### Treatment of Subjects Suffering From Psoriasis

Subject 4

Anamnesis: 32-year-old male subject with psoriatic lesions on arms and knees since age 20. During the last 5 years the lesions had aggravated, becoming larger and flakier in appearance, accompanied by increased pruritus. This change in the lesions coincided with him getting a job in a stressful workplace, namely the stock market.

Previous treatment(s): The subject had used corticosteroids such as mometasone furoate, prednicarbate and most recently calcitriol, always supplementing the treatment with moisturisers and emollients. At first the subject responded well to the treatment, but after a while the treatment was no longer as effective and he had more frequent relapses.

Outcome: The subject began administrating the night cream according example 1 once a day in the evening prior to sleep and the day cream according example 2 once a day in the morning. After a week, the subject reported that some improvement had been achieved and that the treatment was working well according to his opinion. The subject came back three weeks later to get more of the night and day cream and told that the lesions had markedly decreased and that the itching and flaking had ceased. However, skin pigmentation was reported to still not be normal. The reported changes were checked by the experimenter.

After four months the subject reported that he was very pleased with the treatment and that he was beginning to “forget about” the psoriasis, he no longer experienced pruritus and his major lesions (knees and elbows) were by then practically imperceptible. By now, the subject was still using the day cream every day and but was only applying the night one when a small lesion appeared.

Conclusion: Despite reporting to not being helped by corticosteroid treatment lately, the subject experienced efficient symptom relief with the night cream

Subject 5

Anamnesis: 60-year-old female subject with guttate psoriasis on her upper trunk and back. Also with localised plaque lesions on her elbows and knees. The subject was diagnosed to suffer from severe psoriasis.

Previous treatment(s): The subject had undergone numerous treatments prescribed by dermatologists consisting, as usual, of a topical treatment combining corticosteroids and tar extracts, supplemented by moisturisers and emollients. The subject had also used the latest generation pimecrolimus corticosteroid.

Outcome: The subject began administrating the night cream according example 1 once a day in the evening prior to sleep and the day cream according example 2 once a day in the morning. The subject had stopped medicating two months previously. Already after 4 days of treatment the subject reported a good feeling for the treatment. Two months later the subject's psoriasis had improved substantially. The lesions at elbows, knees, upper trunk and back were all abating. Further, it could be seen that the epidermis was largely back to normal, although some darker pigmentation still remained, especially where the most severe lesions had been present.

Conclusion: The subject thus reported a significantly improved effect over conventional treatment of combined use of corticosteroids and tar extracts, supplemented by moisturisers and emollients.

Subject 6

Anamnesis: 45-year-old male subject with psoriasis since age 18, with moderate to severe plaque lesions on his ears and knees and a lesion measuring about 15 cm in diameter on his upper back. The subject complained especially about pruritus and major desquamation. Typically the subject reported to improve during the summer. However, the same symptoms reappeared every winter.

Previous treatment(s): During the recent years the dermatologist had prescribed various magistral formulas containing keratolytics, corticosteroids, tar derivatives, etc. Some of these formulas were difficult to apply and had unpleasant organoleptic properties. This subject reported being tired of trying conventional and new treatments, which he was never happy with in the end.

Outcome: The subject was offered to try out the day and night creams according to example 1 and 2. The subject, previously having experienced side effect of corticosteroids, was reluctant to try yet another corticosteroid based treatment. However, as the subject was told that the side effects commonly seen with corticosteroids were significantly reduced with the present formulations, the subject agreed to try the formulations for a month. The subject thus began administrating the night cream according example 1 once a day in the evening prior to sleep and the day cream according example 2 once a day in the morning.

After a month, the subject reported that the lesions had improved considerably. The pruritus and desquamation had ceased, although the subjects pigmentation was still red. After continued treatment for a year, the subjects' psoriasis was in remission and the psoriasis was virtually cured in terms of lesions.

Conclusion: The subject thus reported an improved effect over conventional treatment of combined use of corticosteroids and tar extracts, supplemented by moisturisers and emollients; and importantly essentially no side-effects from the corticosteroid of the night cream.

1. A dermal composition comprising:
a tar extract;
a corticosteroid;
an antipruritic;
an antibiotic;
urea;
an absorption base;
water;
lanolin; and
propylene glycol.

2. The composition according to claim 1, wherein:
the tar extract is saponified coal tar;
the antipruritic is an antihistamine; and/or
the antibiotic is an anti-fungal agent

3. The composition according to claim 2, wherein
the corticosteroid is selected from the group consisting of: clobetasol propionate, betamethasone dipropionate, halobetasol propionate, fluocinonide, diflorasone diacetate, mometasone furoate, halcinonide, and desoximetasone;
the antihistamine is selected from the group consisting of: chlorpheniramine, diphenhydramine, thonzylamine, mepyramine, tenalidine, tripelenamine, chloropyramine, promethazine, tolpropamine, isothipendyl, and chlorphenoxamine; and/or
the anti-fungal agent is selected from the group consisting of: tolnaftate, amorolfin, butenafine, naftifine, and terbinafine.

4. The composition according to claim 3, wherein the corticosteroid is clobetasol propionate.

5. The composition according to claim 1, wherein the absorption base is a self emulsifying oil/water absorption base.

6. The composition according to claim 1, wherein the composition further comprises glycerin, a vegetable oil, such as sweet almond oil, ammonium lactate, and/or a preservative.

7. The composition according to claim 1, wherein the composition comprises:
5 to 10 wt % of the tar extract;
0.01 to 2 wt % of the corticosteroid;
0.01 to 2 wt % of the antipruritic;
0.5 to 5 wt % of the antibiotic;
5 to 15 wt % urea;
10 to 30 wt % of the absorption base;
30 to 50 wt % water; 1 to 5 wt % lanolin; and
1 to 10 wt % propylene glycol;

8. The composition according to claim 7, wherein the composition further comprises:
1 to 10 wt % of a vegetable oil;
5 to 15 wt % of an aqueous solution of ammonium lactate; and/or
1 to 10 wt % glycerin.

9. The composition according to claim 1, wherein the composition further comprises a preservative.

10. The composition according to claim 9, wherein said preservative is a paraben.

11. A method of treating or soothing psoriasis lesions, comprising the step of applying a composition according to claim 1 to areas of the skin affected by psoriasis.

12. The method according to claim 11, wherein the composition is applied once a day in the evening prior to sleep.

13. A kit comprising two distinct compositions, wherein the first composition is a composition according to claim 1, and the second composition is a composition comprising aqueous hyaluronic acid, an absorption base, and vegetable oil.

14. The kit according to claim 13, wherein the second composition further comprises vitamin E, and optionally a fragrance, such as peach essence, and wherein said vegetable oil comprises almond oil, argan oil, and/or macadamia oil.

15. A method of treating or soothing psoriasis lesions, comprising the step of separately applying the two compositions according to claim 13 to a subject suffering from psoriasis; wherein
the first composition is applied to areas of the skin affected by psoriasis; and
the first composition is to be administered once a day in the evening prior to sleep and the second composition is administered once a day in the morning.

16. The composition according to claim 10, wherein said preservative is propyl 4-hydroxybenzoate or methl 4-hydroxybenzoate.
